(19) United States
(12) Patent Application Publication
(10) Pub. No.: US 2026/0138975 A1
(43) Pub. Date: May 21, 2026

(54) METHOD FOR PRODUCING SULFONE DERIVATIVE USING HALOACETIC ACID

(71) Applicant: KUMIAI CHEMICAL INDUSTRY CO., LTD., Tokyo (JP)

(72) Inventor: Shinki TANI, Tokyo (JP)

(21) Appl. No.: 18/876,731

(22) PCT Filed: Jun. 19, 2023
(86) PCT No.: PCT/JP2023/022520

§ 371 Date: Oct. 29, 2025

(30) Foreign Application Priority Data
Jun. 21, 2022 (JP) 2022-099282

(51) Int. Cl. C07D 413/12; A01N 43/74; A01P 13/00

(52) U.S. Cl. CPC C07D 413/12; A01N 43/74; A01P 13/00

(57) ABSTRACT

The present invention provides an industrially desirable production method for a sulfone derivative that is useful as a herbicide, and an intermediate thereof.

### TECHNICAL FIELD

The present invention relates to a process for producing a sulfone derivative that is, a compound of the following formula (2) useful as a herbicide,

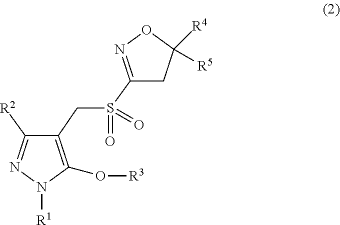

wherein R¹, R², R³, R⁴ and R⁵ are as described herein.

### BACKGROUND ART

It is known that sulfone derivatives of the above formula (2) have a herbicidal activity as disclosed in WO2002/062770 A1 (Patent Document 1). Among them, a compound of the formula (2-a) (pyroxasulfone) is well known as a superior herbicide.

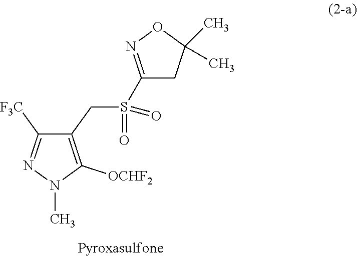 [SMILES: Cn1nc(C(F)(F)F)c(CS(=O)(=O)C2=NOC(C)(C)C2)c1OC(F)F]

As a process for producing the compound of the formula (2), a process by the oxidation of a sulfide derivative, i.e., a compound of the following formula (1) has been known, which is shown below.

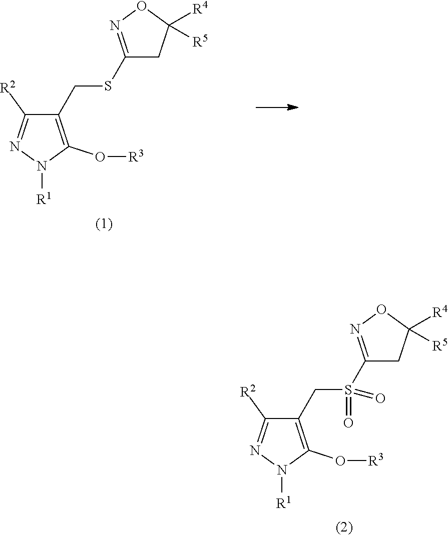

As shown in the following scheme, a process for producing 3-(5-difluoromethoxy-1-methyl-3-trifluoromethyl-1H-pyrazol-4-ylmethanesulfonyl)-5,5-dimethyl-2-isoxazoline (2-a) (pyroxasulfone) by oxidizing 3-(5-difluoromethoxy-1-methyl-3-trifluoromethyl-1H-pyrazol-4-ylmethylthio)-5,5-dimethyl-2-isoxazoline (1-a) (ISFP) with m-chloroperoxybenzoic acid (mCPBA) is disclosed in Reference Example 3 of WO2004/013106 A1 (Patent Document 2).

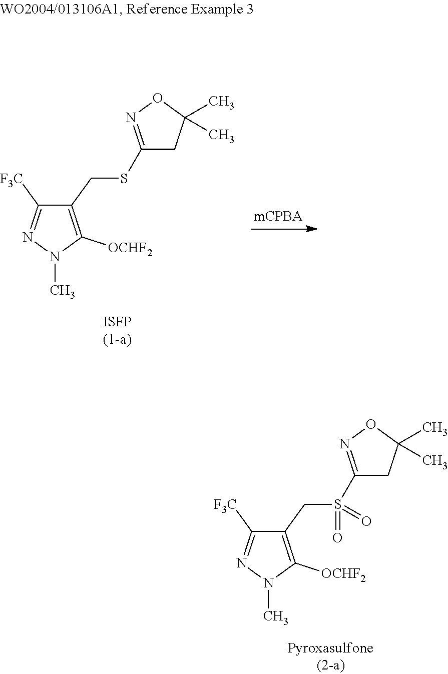 [SMILES: Cn1nc(C(F)(F)F)c(CSC2=NOC(C)(C)C2)c1OC(F)F ; Cn1nc(C(F)(F)F)c(CS(=O)(=O)C2=NOC(C)(C)C2)c1OC(F)F]

In a process for producing the compound of the formula (2) from the compound of the formula (1), m-chloroperbenzoic acid (mCPBA) described in WO2004/013106 A1 (Patent Document 2) is expensive for industrial use, and a problem of handling and waste occurs. Therefore, the process for producing described in WO2004/013106 A1 (Patent Document 2) is not practical for production on an industrial scale.

In addition, in the process for producing the compound of the formula (2) (sulfone derivative: SO₂ derivative) from the compound of the formula (1) (sulfide derivative: S derivative), there is a possibility that the reaction stops at a sulfoxide derivative (SO derivative) i.e., a compound of the following formula (3) that is an intermediate of the oxidation reaction,

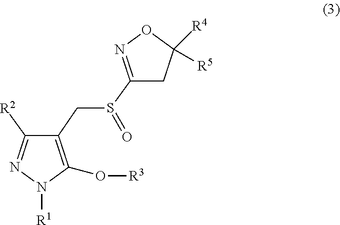

Wherein R¹, R², R³, R⁴ and R⁵ are as described herein. Therefore, the compound of the formula (3) sometimes remains in the product as a by-product. The compound of formula (3) mixed in products such as herbicides leads to the possible reduced quality and damage to crops. However, because the physical and chemical properties of the compound of the formula (3) are very similar to those of the compound of the formula (2), it is difficult to separate the compound of the formula (3) to purify the compound of the formula (2). Therefore, regarding the process for producing the compound of the formula (2) from the compound of the formula (1), a production process in which the oxidation reaction sufficiently proceeds and the amount of the compound of the formula (3) in the product is sufficiently small has been required.

WO2021/002484 A9 (Patent Document 5) describes a process for producing pyroxasulfone. This process is a superior process that has solved the above-described problems. On the other hand, there is still room for improvement in this process because a transition metal is used.

JP2013-512201 A (Patent Literature 6) describes a process for producing pyroxasulfone using acetic acid in Example 9C. The above described problem is, however, not solved by this process, and in addition, this process is desired to be improved because a transition metal is used therein.

JP2013-512201 A (Patent Literature 6) corresponds to US2012/264947 A1 (Patent Literature 7).

CN111574511 A (Patent Document 8) describes a production process without using a transition metal in Example 4. The yield described therein is, however, low, and the process is not reproducible.

### PRIOR ART PATENT DOCUMENT

Patent Document 1: WO2002/062770 A1
Patent Document 2: WO2004/013106 A1
Patent Document 3: WO2005/095352 A1
Patent Document 4: WO2005/105755 A1
Patent Document 5: WO2021/002484 A1
Patent Document 6: JP2013-512201 A
Patent Document 7: US2012/264947 A1
Patent Document 8: CN111574511 A

### SUMMARY OF INVENTION

### Technical Problem

The object of the present invention provides a process for producing a compound of the formula (2) from a compound of the formula (1), that is, an industrially favorable production process in which the ratio of a compound of the formula (3) in the product is sufficiently low, which has an excellent yield and is advantageous for production on an industrial scale.

The another object of the present invention provides an environmentally friendly process for producing a compound of the formula (2).

### Solution to Problem

As a result of earnest study, the present inventors have found, as described below, that a compound of the formula (2) can be efficiently produced by reacting a compound of the formula (1) with an oxidizing agent in the presence of a haloacetic acid without using a transition metal as a catalyst. Based on this finding, the present inventors have accomplished the present invention.

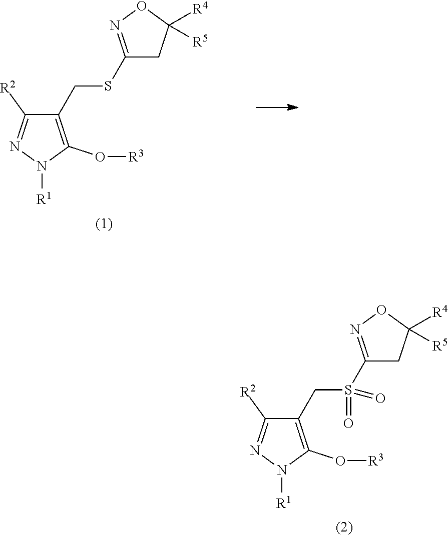

wherein R¹, R², R³, R⁴ and R⁵ are as described herein.

### Advantageous Effects of Invention

The present invention provides a novel process for producing a compound of the formula (2) which has an excellent yield, and is environmentally friendly because no transition metal is used therein. Accordingly, the present invention contributes to sustainability.

The present invention also provides a process for producing a compound of the formula (2) (sulfone derivative: SO₂ derivative) from a compound of the formula (1) (sulfide derivative: S derivative), in which the ratio of a compound of the formula (3) (sulfoxide derivative: SO derivative) in a product is sufficiently low, which has and an excellent yield and is advantageous for production on an industrial scale. In the compound of the formula (2) produced by the process of the present invention, the amount of the compound of the formula (3), which can be a cause of reduced quality as a herbicide and damage to crops, is sufficiently small, and hence this compound is useful as a herbicide.

The process of the present invention can be implemented on a large scale using low-cost materials, and is superior in economic efficiency, and is suitable for production on an industrial scale.

### DESCRIPTION OF EMBODIMENTS

The present invention will now be described in detail.

In one aspect, the present invention is as follows:
[I-1] A process for producing a compound of formula (2), comprising reacting a compound of formula (1) with an oxidizing agent in the presence of a haloacetic acid and in the absence of a transition metal:

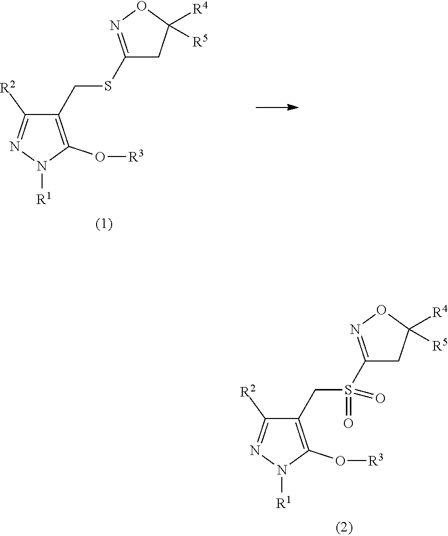

wherein
R¹, R², and R³ are each independently a (C1-C6)alkyl optionally substituted with one or more substituents; a (C3-C6)cycloalkyl optionally substituted with one or more substituents; a (C2-C6)alkenyl optionally substituted with one or more substituents; a (C2-C6)alkynyl optionally substituted with one or more substituents; or a (C6-C10)aryl optionally substituted with one or more substituents, and
R⁴ and R⁵ are each independently a (C1-C6)alkyl optionally substituted with one or more substituents; a (C3-C6)cycloalkyl optionally substituted with one or more substituents; a (C2-C6)alkenyl optionally substituted with one or more substituents; a (C2-C6)alkynyl optionally substituted with one or more substituents; a (C1-C6)alkoxy optionally substituted with one or more substituents; or a (C6-C10)aryl optionally substituted with one or more substituents, or
R⁴ and R⁵, together with a carbon atom to which they are attached, form a 4- to 12-membered carbocyclic ring, wherein the carbocyclic ring is optionally substituted with one or more substituents.
[I-2] The process according to [I-1], wherein the haloacetic acid is selected from monochloroacetic acid, dichloroacetic acid, and trichloroacetic acid.
[I-3] The process according to [I-1], wherein the haloacetic acid is one or more (preferably one or two, and more preferably one) haloacetic acids selected from monochloroacetic acid, dichloroacetic acid, and trichloroacetic acid.
[I-4] The process according to [I-1], wherein the haloacetic acid is dichloroacetic acid.
[I-5] The process according to [I-1], wherein the haloacetic acid is trichloroacetic acid.
[I-6] The process according to any one of [I-1] to [I-5], wherein the amount of the haloacetic acid used is 1 mol or more based on 1 mol of the compound of the formula (1).
[I-7] The process according to any one of [I-1] to [I-5], wherein the amount of the haloacetic acid used is 3 mol or more based on 1 mol of the compound of the formula (1).
[I-8] The process according to any one of [I-1] to [I-5], wherein the amount of the haloacetic acid used is 5 mol or more based on 1 mol of the compound of the formula (1).
[I-9] The process according to any one of [I-1] to [I-5], wherein the amount of the haloacetic acid used is 8 mol or more based on 1 mol of the compound of the formula (1).
[I-10] The process according to any one of [I-1] to [I-5], wherein the amount of the haloacetic acid used is 10 mol or more based on 1 mol of the compound of the formula (1).
[I-11] The process according to any one of [I-1] to [I-5], wherein the amount of the haloacetic acid used is 15 mol or more based on 1 mol of the compound of the formula (1).
[I-12] The process according to any one of [I-1] to [I-11], wherein the amount of the haloacetic acid used is 100 mol or less based on 1 mol of the compound of the formula (1).
[I-13] The process according to any one of [I-1] to [I-11], wherein the amount of the haloacetic acid used is 60 mol or less based on 1 mol of the compound of the formula (1).
[I-14] The process according to any one of [I-1] to [I-11], wherein the amount of the haloacetic acid used is 35 mol or less based on 1 mol of the compound of the formula (1).
[I-15] The process according to any one of [I-1] to [I-11], wherein the amount of the haloacetic acid used is 25 mol or less based on 1 mol of the compound of the formula (1).
[I-16] The process according to any one of [I-1] to [I-11], wherein the amount of the haloacetic acid used is 20 mol or less based on 1 mol of the compound of the formula (1).
[I-17] The process according to any one of [I-1] to [I-16], wherein the amount of the haloacetic acid used is 0.1 liters or more based on 1 mol of the compound of the formula (1).
[I-18] The process according to any one of [I-1] to [I-16], wherein the amount of the haloacetic acid used is 0.3 liters or more based on 1 mol of the compound of the formula (1).
[I-19] The process according to any one of [I-1] to [I-16], wherein the amount of the haloacetic acid used is 0.4 liters or more based on 1 mol of the compound of the formula (1).
[I-20] The process according to any one of [I-1] to [I-16], wherein the amount of the haloacetic acid used is 0.8 liters or more based on 1 mol of the compound of the formula (1).
[I-21] The process according to any one of [I-1] to [I-16], wherein the amount of the haloacetic acid used is 1.2 liters or more based on 1 mol of the compound of the formula (1).
[I-22] The process according to any one of [I-1] to [I-16], wherein the amount of the haloacetic acid used is 1.3 liters or more based on 1 mol of the compound of the formula (1).
[I-23] The process according to any one of [I-1] to [I-22], wherein the amount of the haloacetic acid used is 10 liters or less based on 1 mol of the compound of the formula (1).
[I-24] The process according to any one of [I-1] to [I-22], wherein the amount of the haloacetic acid used is 5.0 liters or less based on 1 mol of the compound of the formula (1).
[I-25] The process according to any one of [I-1] to [I-22], wherein the amount of the haloacetic acid used is 3.0 liters or less based on 1 mol of the compound of the formula (1).
[I-26] The process according to any one of [I-1] to [I-22], wherein the amount of the haloacetic acid used is 2.0 liters or less based on 1 mol of the compound of the formula (1).
[I-27] The process according to any one of [I-1] to [I-26], wherein the reaction is performed in the presence of an organic solvent other than the haloacetic acid.
[I-28] The process according to any one of [I-1] to [I-26], wherein the reaction is performed in the presence of an organic solvent other than the haloacetic acid, and the organic solvent is selected from aromatic hydrocarbon derivatives, halogenated aliphatic hydrocarbons, carboxylic acids, nitriles, carboxylic acid esters, ethers, ketones, amides, ureas, and sulfones.
[I-29] The process according to any one of [I-1] to [I-26], wherein the reaction is performed in the presence of an organic solvent other than the haloacetic acid, and the organic solvent is one or more (preferably one or two, and more preferably one) organic solvents selected from aromatic hydrocarbon derivatives, halogenated aliphatic hydrocarbons, carboxylic acids, nitriles, carboxylic acid esters, ethers, ketones, amides, ureas, and sulfones.
[I-30] The process according to any one of [I-1] to [I-26], wherein the reaction is performed in the presence of an organic solvent other than the haloacetic acid, and the organic solvent is selected from nitriles, carboxylic acids, and amides.
[I-31] The process according to any one of [I-1] to [I-26], wherein the reaction is performed in the presence of an organic solvent other than the haloacetic acid, and the organic solvent is one or two (preferably one) organic solvents selected from nitriles, carboxylic acids, and amides.
[I-32] The process according to any one of [I-1] to [I-26], wherein the reaction is performed in the presence of an organic solvent other than the haloacetic acid, and the organic solvent is acetonitrile.
[I-33] The process according to any one of [I-1] to [I-26], wherein the reaction is performed in the presence of an organic solvent, and the organic solvent is an organic solvent excluding alcohols.
[I-34] The process according to any one of [I-1] to [I-26], wherein the reaction is performed in the presence of an organic solvent, and the organic solvent is an organic solvent excluding (C1-C6)alcohols.
[I-35] The process according to any one of [I-1] to [I-26], wherein the reaction is performed in the presence of an organic solvent, and the organic solvent is an organic solvent excluding (C1-C4)alcohols.
[I-36] The process according to any one of [I-1] to [I-35], wherein the amount of the organic solvent other than the haloacetic acid used in the reaction is 0.3 liters or more based on 1 mol of the compound of the formula (1).
[I-37] The process according to any one of [I-1] to [I-35], wherein the amount of the organic solvent other than the haloacetic acid used in the reaction is 0.5 liters or more based on 1 mol of the compound of the formula (1).
[I-38] The process according to any one of [I-1] to [I-35], wherein the amount of the organic solvent other than the haloacetic acid used in the reaction is 3.0 liters or less based on 1 mol of the compound of the formula (1).
[I-39] The process according to any one of [I-1] to [I-35], wherein the amount of the organic solvent other than the haloacetic acid used in the reaction is 2.0 liters or less based on 1 mol of the compound of the formula (1).
[I-40] The process according to any one of [I-1] to [I-26], wherein the reaction is performed in the absence of an organic solvent other than the haloacetic acid.
[I-41] The process according to any one of [I-1] to [I-40], wherein the reaction is performed at 10° C. to 100° C.
[I-42] The process according to any one of [I-1] to [I-40], wherein the reaction is performed at 30° C. to 80° C.
[I-43] The process according to any one of [I-1] to [I-40], wherein the reaction is performed at 40° C. to 80° C.
[I-44] The process according to any one of [I-1] to [I-40], wherein the reaction is performed at 40° C. to 65° C.
[I-45] The process according to any one of [I-1] to [I-44], wherein the reaction is performed for 30 minutes or more.
[I-46] The process according to any one of [I-1] to [I-44], wherein the reaction is performed for 1 hour or more.
[I-47] The process according to any one of [I-1] to [I-46], wherein the reaction is performed for 48 hours or less.
[I-48] The process according to any one of [I-1] to [I-46], wherein the reaction is performed for 12 hours or less.
[I-49] The process according to any one of [I-1] to [I-46], wherein the reaction is performed for 6 hours or less.
[I-50] The process according to any one of [I-1] to [I-46], wherein the reaction is performed for 4 hours or less.
[I-51] The process according to any one of [I-1] to [I-46], wherein the reaction is performed for 2 hours or less.
[I-52] The process according to [I-1] to [I-51], wherein the reaction is performed in the presence of a water solvent.
[I-53] The process according to any one of [I-1] to [I-52], wherein the amount of the water solvent used in the reaction is 0.05 liters or more based on 1 mol of the compound of the formula (1).
[I-54] The process according to any one of [I-1] to [I-52], wherein the amount of the water solvent used in the reaction is 0.1 liters or more based on 1 mol of the compound of the formula (1).
[I-55] The process according to any one of [I-1] to [I-54], wherein the amount of the water solvent used in the reaction is 0.5 liters or less based on 1 mol of the compound of the formula (1).
[I-56] The process according to any one of [I-1] to [I-54], wherein the amount of the water solvent used in the reaction is 0.3 liters or less based on 1 mol of the compound of the formula (1).
[I-57] The process according to any one of [I-1] to [I-56], wherein the oxidizing agent is hydrogen peroxide, a persulfate, or a hydrogen persulfate.
[I-58] The process according to any one of [I-1] to [I-56], wherein the oxidizing agent is hydrogen peroxide.
[I-59] The process according to any one of [I-1] to [I-56], wherein the oxidizing agent is a 10 to 70 wt % aqueous hydrogen peroxide solution.
[I-60] The process according to any one of [I-1] to [I-56], wherein the oxidizing agent is a 25 to 65 wt % aqueous hydrogen peroxide solution.
[I-61] The process according to any one of [I-1] to [I-60], wherein the amount of the oxidizing agent used is 2 to 8 mol based on 1 mol of the compound of the formula (1).
[I-62] The process according to any one of [I-1] to [I-60], wherein the amount of the oxidizing agent used is 2 to 5 mol based on 1 mol of the compound of the formula (1).
[I-63] The process according to any one of [I-1] to [I-60], wherein the amount of the oxidizing agent used is 2.5 to 4 mol based on 1 mol of the compound of the formula (1).
[I-64] The process according to any one of [I-1] to [I-63],
   wherein in the formula (1) and the formula (2),
   R¹ is a (C1-C4)alkyl,
   R² is a (C1-C4)perfluoroalkyl,
   R³ is a (C1-C4)alkyl optionally substituted with 1 to 9 fluorine atoms, and
   R⁴ and R⁵ are each independently a (C1-C4)alkyl.
[I-65] The process according to any one of [I-1] to [I-63], wherein
   in the formulas (1) and (2),
   R¹ is methyl,
   R² is trifluoromethyl,
   R³ is difluoromethyl, and
   R⁴ and R⁵ are methyl.

In another aspect, the present invention is as follows.
[II-1] A process for producing a compound of formula (2), comprising reacting a compound of formula (1) with an oxidizing agent in the presence of a haloacetic acid and in the absence of a transition metal:

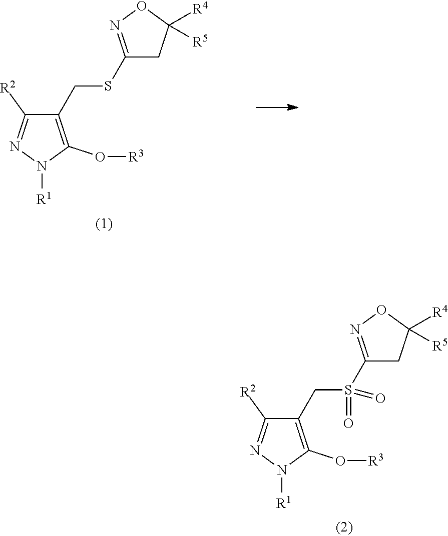

wherein in the formula (1) and the formula (2),
R¹, R², and R³ are each independently a (C1-C6)alkyl optionally substituted with one or more substituents; a (C3-C6)cycloalkyl optionally substituted with one or more substituents; a (C2-C6)alkenyl optionally substituted with one or more substituents; a (C2-C6)alkynyl optionally substituted with one or more substituents; or a (C6-C10)aryl optionally substituted with one or more substituents, and
R⁴ and R⁵ are each independently a (C1-C6)alkyl optionally substituted with one or more substituents; a (C3-C6)cycloalkyl optionally substituted with one or more substituents; a (C2-C6)alkenyl optionally substituted with one or more substituents; a (C2-C6)alkynyl optionally substituted with one or more substituents; a (C1-C6)alkoxy optionally substituted with one or more substituents; or a (C6-C10)aryl optionally substituted with one or more substituents, or
R⁴ and R⁵, together with a carbon atom to which they are attached, form a 4- to 12-membered carbocyclic ring, wherein the carbocyclic ring is optionally substituted with one or more substituents.
[II-2] The process according to [II-1], wherein the haloacetic acid is selected from monochloroacetic acid, dichloroacetic acid, and trichloroacetic acid.
[II-3] The process according to [II-1], wherein the haloacetic acid is dichloroacetic acid.
[II-4] The process according to [II-1], wherein the haloacetic acid is trichloroacetic acid.
[II-5] The process according to any one of [II-1] to [II-4], wherein the oxidizing agent is hydrogen peroxide.
[II-6] The process according to any one of [II-1] to [II-5],
   wherein in the formula (1) and the formula (2),
   R¹ is a (C1-C4)alkyl,
   R² is a (C1-C4)perfluoroalkyl,
   R³ is a (C1-C4)alkyl optionally substituted with 1 to 9 fluorine atoms, and
   R⁴ and R⁵ are each independently a (C1-C4)alkyl.
[II-7] The process according to any one of [II-1] to [II-5],
   wherein in the formula (1) and the formula (2),
   R¹ is methyl,
   R² is trifluoromethyl,
   R³ is difluoromethyl, and
   R⁴ and R⁵ are methyl.

The symbols and terms described in the present description will be explained.

Herein, the following abbreviations and prefixes may be used, and their meanings are as follows:
Me: methyl
Et: ethyl
Pr, n-Pr and Pr-n: propyl (i.e., normal propyl)
i-Pr and Pr-i: isopropyl
Bu, n-Bu and Bu-n: butyl (i.e., normal butyl)
s-Bu and Bu-s: sec-butyl (i.e., secondary butyl)
i-Bu and Bu-i: isobutyl
t-Bu and Bu-t: tert-butyl (i.e., tertiary butyl)
Ph: phenyl
n-: normal
s- and sec-: secondary
i- and iso-: iso
t- and tert-: tertiary
c- and cyc-: cyclo
o-: ortho
m-: meta
p-: para

The term “nitro” means the substituent “—NO₂”.

The term “cyano” or “nitrile” means the substituent “—CN”.

The term “hydroxy” means the substituent “—OH”.

The term “amino” means the substituent “—NH₂”.

(Ca-Cb) means that the number of carbon atoms is a to b. For example, “(C1-C4)” in “(C1-C4)alkyl” means that the number of the carbon atoms in the alkyl is 1 to 4, and “(C2-C5)” means that the number of the carbon atoms in the alkyl is 2 to 5. “(Ca-Cb)” meaning the number of carbon atoms may be written as “Ca-Cb” without parentheses. Thus, for example, “C1-C4” in “C1-C4 alkyl” means that the number of the carbon atoms in the alkyl is 1 to 4.

Herein, it is to be interpreted that generic terms such as “alkyl” include both the straight chain and the branched chain such as butyl and tert-butyl. Meanwhile, for example, the specific term “butyl” refers to straight “normal butyl”, and does not refer to branched “tert-butyl”. Branched chain isomers such as “tert-butyl” are referred to specifically when intended.

Examples of the halogen atom include fluorine atom, chlorine atom, bromine atom and iodine.

The (C1-C6)alkyl means a straight or branched alkyl having 1 to 6 carbon atoms. Examples of the (C1-C6)alkyl include, but are not limited to, methyl, ethyl, propyl, isopropyl, butyl, sec-butyl, isobutyl, tert-butyl, pentyl and hexyl.

The (C1-C4)alkyl means a straight or branched alkyl having 1 to 4 carbon atoms. Examples of the (C1-C4)alkyl include, appropriate examples of the examples of the (C1-C6)alkyl above-mentioned.

The (C3-C6)cycloalkyl means a cycloalkyl having 3 to 6 carbon atoms. Examples of the (C3-C6)cycloalkyl are cyclopropyl, cyclobutyl, cyclopentyl and cyclohexyl.

The (C2-C6)alkenyl means a straight or branched alkenyl having 2 to 6 carbon atoms. Examples of the (C2-C6)alkenyl include, but are not limited to, vinyl, 1-propenyl, isopropenyl, 2-propenyl, 1-butenyl, 1-methyl-1-propenyl, 2-methyl-1-propenyl, 2-butenyl, 3-butenyl, 1,3-butadienyl, 1-pentenyl and 1-hexenyl.

The (C2-C6)alkynyl means a straight or branched alkynyl having 2 to 6 carbon atoms. Examples of the (C2-C6)alkynyl include, but are not limited to, ethynyl, 1-propynyl, 2-propynyl, 1-butynyl, 1-methyl-2-propynyl, 2-butynyl, 3-butynyl, 1-pentynyl and 1-hexynyl.

Examples of the (C6-C10)aryl are phenyl, 1-naphthyl and 2-naphthyl.

The (C1-C6)haloalkyl means a straight or branched alkyl having 1 to 6 carbon atoms which is substituted with 1 to 13 same or different halogen atoms, wherein the halogen atoms have the same meaning as defined above. Examples of the (C1-C6)haloalkyl include, but are not limited to, fluoromethyl, chloromethyl, bromomethyl, difluoromethyl, dichloromethyl, trifluoromethyl, trichloromethyl, chlorodifluoromethyl, bromodifluoromethyl, 2-fluoroethyl, 1-chloroethyl, 2-chloroethyl, 2,2,2-trifluoroethyl, pentafluoroethyl, 3-fluoropropyl, 3-chloropropyl, 2-chloro-1-methylethyl, 2,2,3,3,3-pentafluoropropyl, 2,2,2-trifluoro-1-trifluoromethylethyl, heptafluoropropyl, 1,2,2,2-tetrafluoro-1-trifluoromethylethyl, 4-fluorobutyl, 4-chlorobutyl, 2,2,3,3,4,4,4-heptafluorobutyl, nonafluorobutyl, 1,1,2,3,3,3-hexafluoro-2-trifluoromethylpropyl, 2,2,2-trifluoro-1,1-di(trifluoromethyl)ethyl, undecafluoropentyl and tridecafluorohexyl.

The (C1-C4)perfluoroalkyl means a straight or branched alkyl having 1 to 4 carbon atoms, wherein all hydrogen atoms are substituted with fluorine atoms. Examples of the (C1-C4)perfluoroalkyl are trifluoromethyl (i.e., —CF₃), pentafluoroethyl (i.e., —CF₂CF₃), heptafluoropropyl (i.e., —CF₂CF₂CF₃), 1,2,2,2-tetrafluoro-1-trifluoromethylethyl (i.e., —CF(CF₃)₂), nonafluorobutyl, (i.e., —CF₂CF₂CF₂CF₃), 1,2,2,3,3,3-hexafluoro-1-trifluoromethylpropyl (i.e., —CF(CF₃)CF₂CF₃), 1,1,2,3,3,3-hexafluoro-2-trifluoromethylpropyl (i.e., —CF₂CF(CF₃)₂) and 2,2,2-trifluoro-1,1-di(trifluoromethyl) ethyl (i.e., —C(CF₃)₃).

The (C1-C6)alkoxy means a (C1-C6)alkyl-O—, wherein the (C1-C6)alkyl moiety has the same meaning as defined above. Examples of the (C1-C6)alkoxy include, but are not limited to, methoxy, ethoxy, propoxy, isopropoxy, butoxy, sec-butoxy, isobutoxy, tert-butoxy, pentyloxy, isopentyloxy, neopentyloxy and hexyloxy.

The (C1-C6)alcohol means a (C1-C6)alkyl-OH, wherein the (C1-C6)alkyl moiety has the same meaning as defined above. Examples of the (C1-C6)alcohol include, but are not limited to, methanol, ethanol, propanol (i.e., 1-propanol), 2-propanol, butanol (i.e., 1-butanol), sec-butanol, isobutanol, tert-butanol, pentanol (i.e., 1-pentanol), sec-amyl alcohol, 3-pentanol, 2-methyl-1-butanol, isoamyl alcohol, tert-amyl alcohol, hexanol (i.e., 1-hexanol) and cyclohexanol. Polyols having 1 to 6 carbons (e.g., diols and triols) such as ethylene glycol, propylene glycol and glycerol are equivalents of (C1-C6)alcohols.

Examples of the 4- to 12-membered carbocyclic ring include, but are not limited to, cyclobutane, cyclopentane, cyclohexane, cycloheptane, cyclooctane, cyclononane, cyclodecane, cycloundecane, and cyclododecane.

Herein, there are no particular limitations on the “substituent(s)” for the phrase “optionally substituted with one or more substituents” as long as they are chemically acceptable and exhibit the effects of the present invention.

Herein, examples of the “substituent(s)” for the phrase “optionally substituted with one or more substituent(s)” include, but are not limited to, one or more substituents (preferably 1 to 3 substituents) selected independently from Substituent Group (a).

Substituent Group (a) is a group consisting of a halogen atom; a nitro group, a cyano group, a hydroxy group, an amino group, (C1-C6)alkyl, (C1-C6)haloalkyl, (C3-C6)cycloalkyl, (C2-C6)alkenyl, (C2-C6)alkynyl, (C1-C6)alkoxy, phenyl and phenoxy.

In addition, one or more substituents (preferably 1 to 3 substituents) selected independently from Substituent Group (a) may each independently be substituted with one or more substituents (preferably 1 to 3 substituents) selected independently from Substituent Group (b). In this context, Substituent Group (b) is the same as Substituent Group (a).

Examples of the “(C1-C6)alkyl optionally substituted with one or more substituents” include, but are not limited to, (C1-C6)haloalkyl, (C1-C4)perfluoroalkyl and (C1-C4)alkyl optionally substituted with 1 to 9 fluorine atoms.

Examples of the (C1-C4)alkyl optionally substituted with 1 to 9 fluorine atoms include, but are not limited to, fluoromethyl (i.e., —CH₂F), difluoromethyl (i.e., —CHF₂), trifluoromethyl (i.e., —CF₃), 2-fluoroethyl, 2,2,2-trifluoroethyl, pentafluoroethyl, 3-fluoropropyl, 2,2,3,3,3-pentafluoropropyl, 2,2,2-trifluoro-1-trifluoromethylethyl, heptafluoropropyl, 1,2,2,2-tetrafluoro-1-trifluoromethylethyl, 4-fluorobutyl, 2,2,3,3,4,4,4-heptafluorobutyl, nonafluorobutyl, 1,1,2,3,3,3-hexafluoro-2 trifluoromethylpropyl and 2,2,2-trifluoro-1,1-di(trifluoromethyl)ethyl.

Herein, the phrase “as described herein” and similar phrases used when referring to substituents (for example, R¹, R², R³, R⁴ and R⁵) incorporate by reference all definitions of the substituents and, if any, all of their examples, preferred examples, more preferred examples, further preferred examples and particularly preferred examples in this specification.

As used herein, the non-limiting term “comprise(s)/comprising” can each optionally be replaced by the limiting phrase “consist(s) of/consisting of”.

Unless otherwise stated, all technical and scientific terms used herein have the same meaning as commonly understood by a person skilled in the art to which the present disclosure belongs.

Unless otherwise indicated, it is understood that numbers used herein to express characteristics such as quantities, sizes, concentrations, and reaction conditions are modified by the term “about”. In some embodiments, disclosed numerical values are interpreted applying the reported number of significant digits and conventional rounding techniques. In some embodiments, disclosed numerical values are interpreted as containing certain errors necessarily resulting from the standard deviation found in their respective testing measurements.

### (Raw Material: Compound of Formula (1))

A compound of the formula (1) is used as a raw material. The compound of the formula (1) may be a known compound, or may be produced from a known compound according to a known process. The preparation of the compound of the formula (1) is described, for example, in Reference Examples 1-1, 1-2, and 1-3 of WO 2004/013106 A1 (Patent Literature 2), Examples 3 to 5 of WO 2005/095352 A1 (Patent Literature 3), and Examples 1 to 5 of WO 2005/105755 A1 (Patent Literature 4).

### (Product; Compound of Formula (2))

The product is a compound of the formula (2) corresponding to the compound of the formula (1) used as a raw material.

In the formula (1) and the formula (2), R¹, R², R³, R⁴, and R⁵ are as defined above.

A particularly preferred specific example of the compound of the formula (2) is as follows:

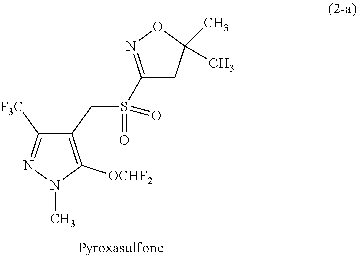 [SMILES: Cn1nc(C(F)(F)F)c(CS(=O)(=O)C2=NOC(C)(C)C2)c1OC(F)F]

An intermediate of an oxidation reaction is a compound of the formula (3) corresponding to the compound of the formula (1) used as the raw material.

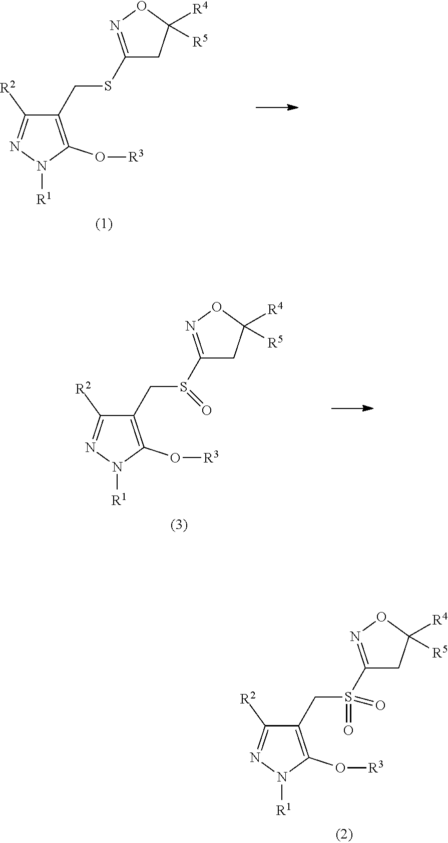

A specific example of the compound of the formula (3) includes the following:

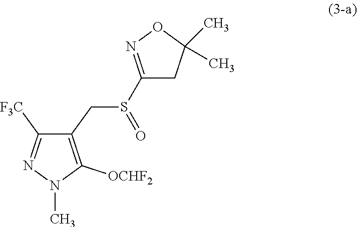 [SMILES: Cn1nc(C(F)(F)F)c(CS(=O)C2=NOC(C)(C)C2)c1OC(F)F]

As described above, in the process of producing the compound of the formula (2) (SO₂ derivative) from the compound of the formula (1) (S derivative), it is desired that the oxidation reaction sufficiently proceeds and the proportion of the compound of the formula (3) (SO derivative) in the product is sufficiently low. For example, in the reaction mixture after the reaction, the ratio of the compound of the formula (3) (SO derivative) is preferably 10% or less, more preferably 5% or less, still more preferably 3% or less, further preferably 2% or less, and further preferably 1% or less.

### (Oxidizing Agent)

A peroxide, an alkali metal persulfate, an ammonium persulfate salt, an alkali metal hydrogen persulfate, a hypochlorite, etc. can be used as the oxidizing agent. Hydrogen peroxide, an alkali metal persulfate, an ammonium persulfate salt, and an alkali metal hydrogen persulfate can be preferably used, hydrogen peroxide and an alkali metal hydrogen persulfate can be more preferably used, and hydrogen peroxide, sodium hydrogen persulfate, sodium persulfate, potassium persulfate, ammonium persulfate, and potassium hydrogen persulfate can be further preferably used, and hydrogen peroxide can be particularly preferably used.

The form of the hydrogen peroxide may be any form as long as the reaction proceeds. The form of the hydrogen peroxide can be suitably selected by a person skilled in the art. In view of safety, danger, economic efficiency, etc., however, preferred examples of the form of the hydrogen peroxide include a 10 to 70 wt % aqueous hydrogen peroxide solution, more preferably a 20 to 70 wt % aqueous hydrogen peroxide solution, still more preferably a 25 to 65 wt % aqueous hydrogen peroxide solution, further preferably a 30 to 65 wt % aqueous hydrogen peroxide solution, and particularly preferably a 30 to 60 wt % aqueous hydrogen peroxide solution. Specific examples of the form of the hydrogen peroxide include, but are not limited to, a 25 wt % aqueous hydrogen peroxide solution, a 30 wt % aqueous hydrogen peroxide solution, a 35 wt % aqueous hydrogen peroxide solution, a 50 wt % aqueous hydrogen peroxide solution and a 60 wt % aqueous hydrogen peroxide solution. The range of the concentration of the hydrogen peroxide may be any combination of the lower limits and the upper limits of the above-described ranges, and such combinations of the lower limits and the upper limits of the ranges are within the scope of the present invention.

The amount of the hydrogen peroxide used may be any amount as long as the reaction proceeds. The amount of the hydrogen peroxide used may be appropriately adjusted by a person skilled in the art. From the viewpoint of yield, suppression of by-products, economic efficiency, safety, etc., however, the lower limit of the amount of the hydrogen peroxide used is, for example, 2 mol or more, 2.3 mol or more, 2.5 mol or more, or 2.8 mol or more based on 1 mol of the compound of the formula (1) (raw material). The upper limit of the amount of the hydrogen peroxide used is, for example, 10 mol or less, 8 mol or less, 7 mol or less, 6 mol or less, 5 mol or less, 4 mol or less, or 3 mol or less based on 1 mol of the compound of the formula (1) (raw material). The amount of the hydrogen peroxide used is within a range of any combination of the lower limits and the upper limits of the ranges described above. The amount of the hydrogen peroxide used is, for example, 2 to 10 mol, preferably 2 to 8 mol, more preferably 2 to 5 mol, further preferably 2 to 4 mol, further preferably 2.5 to 4 mol, and further preferably 2.5 to 3 mol based on 1 mol of the compound of the formula (1) (raw material).

### (In the Absence of Transition Metal)

An oxidation reaction using hydrogen peroxide as an oxidizing agent in the presence of a transition metal catalyst has been reported. In the process of the present invention, however, there is no need for a transition metal catalyst. Accordingly, the term “in the absence of a transition metal” means that a catalyst containing a transition metal catalyst is not used. Accordingly, “in the absence of a transition metal” herein can be optionally replaced by “in the absence of a transition metal catalyst”. Examples of the transition metal not used include, but are not limited to, tungsten, molybdenum, iron, manganese, vanadium, niobium, tantalum, titanium, zirconium, and copper. Examples of the transition metal catalyst not used include, but are not limited to, tungsten catalysts (e.g., sodium tungstate dihydrate), molybdenum catalysts (e.g., ammonium molybdate tetrahydrate), iron catalysts (e.g., iron (III) acetylacetonate, and iron (III) chloride), manganese catalysts (e.g., manganese (III) acetylacetonate), vanadium catalysts (e.g., vanadyl acetylacetonate), niobium catalysts (e.g., sodium niobate), tantalum catalysts (e.g., lithium tantalate), titanium catalysts (e.g., titanium acetylacetonate, and titanium tetrachloride), zirconium catalysts (e.g., zirconium chloride oxide octahydrate) and copper catalysts (e.g., copper (II) acetate, and copper (I) bromide).

### (Haloacetic Acid)

From the viewpoint of yield, suppression of by-products, economic efficiency, etc., examples of the haloacetic acid include, but are not limited to, the following: monochloroacetic acid, dichloroacetic acid, trichloroacetic acid, monobromoacetic acid, bromochloroacetic acid, dibromoacetic acid, bromodichloroacetic acid, dibromochloroacetic acid, tribromoacetic acid, monofluoroacetic acid, difluoroacetic acid, trifluoroacetic acid, and a mixture thereof, preferably monochloroacetic acid, dichloroacetic acid, trichloroacetic acid, and a mixture thereof, and more preferably dichloroacetic acid, trichloroacetic acid, and a mixture thereof.

The haloacetic acid may be used singly or in a combination of two or more kinds thereof in any ratio. The haloacetic acid may be in any form as long as the reaction proceeds. The form of the haloacetic acid can be appropriately selected by a person skilled in the art.

### (Lower Limit of Amount of Haloacetic Acid Used)

The amount of carboxylic acid used is not particularly limited as long as the effects of the present invention are exhibited. From the viewpoint of yield, suppression of by-products, economic efficiency, etc., however, in one embodiment, the lower limit of the amount of the haloacetic acid used is, for example, over 0 (zero) mol, preferably 1 mol or more, 2 mol or more, or 3 mol or more, more preferably 5 mol or more, or 6 mol or more, further preferably 8 mol or more, 9 mol or more, or 10 mol or more, and further preferably 12 mol or more, 15 mol or more, or 18 mol or more based on 1 mol of the compound of the formula (1) (raw material).

From the same viewpoints as described above, in another embodiment, the lower limit of the amount of the haloacetic acid used is, for example, over 0 (zero) liter, preferably 0.1 liters or more, 0.2 liters or more, or 0.3 liters of more, more preferably 0.4 liters or more, 0.5 liters or more, or 0.6 liters or more, further preferably 0.8 liters or more, 0.9 liters or more, or 1.0 liters or more, and further preferably 1.2 liters or more, 1.3 liters or more, or 1.5 liters or more based on 1 mol of the compound of the formula (1) (raw material).

### (Upper Limit of Amount of Haloacetic Acid Used)

From the same viewpoints as described above, in one embodiment, the upper limit of the amount of the haloacetic acid used is, for example, 100 mol or less, preferably 60 mol or less, more preferably 35 mol or less, further preferably 25 mol or less, and further preferably 20 mol or less based on 1 mol of the compound of the formula (1) (raw material).

From the same viewpoints as described above, in another embodiment, the upper limit of the amount of the haloacetic acid used is, for example, 10.0 liters or less, preferably 5.0 liters or less, more preferably 3.0 liters or less, and further preferably 2.0 liters or less based on 1 mol of the compound of the formula (1) (raw material).

### (Range of Amount of Haloacetic Acid Used)

From the same viewpoints as described above, in one embodiment, the range of the amount of the haloacetic acid used is an appropriate and optional combination of any of the lower limits and the upper limits described above. Examples of the combination of the upper limit and the lower limit include, but are not limited to, the following: The amount of the haloacetic acid used is, for example, 3 mol or more and 60 mol or less, preferably 5 mol or more and 35 mol or less, 8 mol or more and 35 mol or less, or 10 mol or more and 35 mol or less based on 1 mol of the compound of the formula (1) (raw material).

From the same viewpoints as described above, in another embodiment, the range of the amount of the haloacetic acid used is an appropriate and optional combination of any of the lower limits and the upper limits described above. Examples of the combination of the upper limit and the lower limit include, but are not limited to, the following: The amount of the haloacetic acid used is, for example, 0.3 liters or more and 5.0 liters or less, preferably 0.5 liters or more and 3.0 liters or less, 0.8 liters or more and 3.0 liters or less, or 1.0 liters or more and 3.0 liters or less based on 1 mol of the compound of the formula (1) (raw material).

The haloacetic acid may be used as a solvent. In this case, the haloacetic acid contributes to the reaction itself, and also functions as a solvent.

The solvent may be in a single layer or may be separated into two layers as long as the reaction proceeds. The reaction system may be homogeneous or inhomogeneous as long as the reaction proceeds. The following has been, however, found through examination of the present invention after accomplishing the present invention. In the present invention, when a haloacetic acid is used, particularly when a haloacetic acid such as dichloroacetic acid, or trichloroacetic acid is used as a solvent, favorable conditions (reaction system) are obtained in the present invention from the viewpoints of the affinity of components of the reaction system, such as a raw material, a product, a reactant solution, and a solvent, and/or the homogeneousness of the reaction system. As a result, for example, a high reaction rate is obtained. A faster reaction is industrially preferable. Besides, it has been found that stirring is easily performed in the present invention.

### (Acid Catalyst)

The oxidation reaction of the present invention may be performed in the presence of an acid catalyst, or in the absence of an acid catalyst. A person skilled in the art can suitably determine whether or not an acid catalyst is used. Here, an acid catalyst refers to an acid other than the haloacetic acid. Examples of the acid catalyst include, but are not limited to, the following: mineral acids such as hydrochloric acid, sulfuric acid, and nitric acid, sulfonic acids such as methanesulfonic acid, trifluoromethanesulfonic acid, benzenesulfonic acid, and p-toluenesulfonic acid, and phosphoric acids such as phosphoric acid, methyl phosphate, ethyl phosphate, and phenyl phosphate, and preferably sulfuric acid, phosphoric acid, or phenyl phosphate, and more preferably sulfuric acid, or phenyl phosphate, and further preferably sulfuric acid. The acid catalyst may be a salt thereof.

The acid catalyst may be used singly or in a combination of two or more kinds thereof in any ratio. The acid catalyst may be in any form as long as the reaction proceeds. For example, examples of sulfuric acid include, but are not limited to, 50% to 98% sulfuric acid, and 50% to 100% sulfuric acid, preferably 90% to 98% sulfuric acid, and 90% to 100% sulfuric acid (concentrated sulfuric acid). The form of the acid catalyst can be appropriately selected by a person skilled in the art. The amount of the acid catalyst used may be any amount as long as the reaction proceeds. The amount of the acid catalyst used can be appropriately adjusted by a person skilled in the art. From the viewpoint of yield, suppression of by-products, economic efficiency, etc., however, when the reaction of the present invention is performed in the presence of an acid catalyst, the amount of the acid catalyst used is, for example, over 0 (zero) and 0.5 mol or less, 0.01 to 0.3 mol, or 0.05 to 0.2 mol based on 1 mol of the compound of the formula (1) (raw material).

### (Phase Transfer Catalyst)

The oxidation reaction of the present invention may be performed in the presence of a phase transfer catalyst, or may be performed in the absence of a phase transfer catalyst. A person skilled in the art can suitably determine whether or not a phase transfer catalyst is used. Examples of the phase transfer catalyst include, but are not limited to, the following: quaternary ammonium salts (e.g., tetrabutylammonium chloride, tetrabutylammonium bromide, tetrabutylammonium iodide, tetrabutylammonium hydrogen sulfate, benzyltrimethylammonium chloride, benzyltrimethylammonium bromide, octyltrimethylammonium chloride, octyltrimethylammonium bromide, trioctylmethylammonium chloride, trioctylmethylammonium bromide, benzyllauryldimethylammonium chloride (benzyldodecyldimethylammonium chloride), benzyllauryldimethylammonium bromide (benzyldodecyldimethylammonium bromide), myristyltrimethylammonium chloride (tetradecyltrimethylammonium chloride), myristyltrimethylammonium bromide (tetradecyltrimethylammonium bromide), benzyldimethylstearylammonium chloride (benzyloctadecyldimethylammonium chloride), and benzyldimethylstearylammonium bromide (benzyloctadecyldimethylammonium bromide)), quaternary phosphonium salts (e.g., tetrabutylphosphonium bromide, tetraoctylphosphonium bromide, and tetraphenylphosphonium bromide), and crown ethers (e.g., 12-crown-4, 15-crown-5, and 18-crown-6). From the viewpoints of yield, suppression of by-products, economic efficiency, etc., examples of the phase transfer catalyst include preferably tetrabutylammonium chloride, tetrabutylammonium bromide, and tetrabutylammonium hydrogen sulfate, and more preferably tetrabutylammonium hydrogen sulfate. Tetrabutylammonium hydrogen sulfate may be abbreviated as TBAHS.

The phase transfer catalyst may be used singly or in a combination of two or more kinds thereof in any ratio. The phase transfer catalyst may be in any form as long as the reaction proceeds. The form of the phase transfer catalyst can be appropriately selected by a person skilled in the art. The amount of the phase transfer catalyst used may be any amount as long as the reaction proceeds. The amount of the phase transfer catalyst used can be appropriately adjusted by a person skilled in the art. From the viewpoints of yield, suppression of by-products, economic efficiency, etc., however, when the reaction of the present invention is performed in the presence of a phase transfer catalyst, the amount of the phase transfer catalyst used is, for example, over 0 (zero) and 0.5 mol or less, 0.01 to 0.3 mol, or 0.05 to 0.2 mol based on 1 mol of the compound of the formula (1) (raw material).

### (Reaction Solvent)

From the viewpoint of allowing the reaction to smoothly proceed, the reaction is preferably performed in the presence of a solvent. The reaction solvent may be any solvent as long as the reaction proceeds. The reaction solvent may be a haloacetic acid, or may be an organic solvent other than a haloacetic acid. In either case, the reaction may be performed in the presence of a water solvent.

Examples of the organic solvent other than a haloacetic acid include, but are not limited to, the following: aromatic hydrocarbon derivatives (e.g., benzene, toluene, xylenes, chlorobenzene, dichlorobenzenes, trichlorobenzenes, and nitrobenzene), halogenated aliphatic hydrocarbons (e.g., dichloromethane, and 1,2-dichloroethane (EDC)), alcohols (e.g., methanol, ethanol, propanol, 2-propanol (2-propanol being also referred to as isopropyl alcohol or isopropanol), butanol, sec-butanol, isobutanol, and tert-butanol (tert-butanol being also referred to as tert-butyl alcohol), pentanol, sec-amyl alcohol, 3-pentanol, 2-methyl-1-butanol, isoamyl alcohol, tert-amyl alcohol, hexanol, and cyclohexanol), nitriles (e.g., acetonitrile, and propionitrile), carboxylic acids (e.g., acetic acid, and propionic acid), carboxylic acid esters (e.g., methyl acetate, ethyl acetate, propyl acetate, isopropyl acetate, butyl acetate and isomers thereof, and pentyl acetate and isomers thereof (in the present invention, an “isomer of butyl acetate” being an equivalent of “butyl acetate”, and an “isomer of pentyl acetate” being an equivalent of “pentyl acetate”)), ethers (e.g., tetrahydrofuran (THF), 1,4-dioxane, diisopropyl ether, dibutyl ether, di-tert-butyl ether, cyclopentyl methyl ether (CPME), methyl tert-butyl ether, 1,2-dimethoxyethane (DME), and diglyme), ketones (e.g., acetone, methyl ethyl ketone (MEK), methyl isopropyl ketone (MIPK), and methyl isobutyl ketone (MIBK)), amides (e.g., N,N-dimethylformamide (DMF), N,N-dimethylacetamide (DMAC), and N-methylpyrrolidone (NMP)), ureas (e.g., N,N′-dimethylimidazolidinone (DMI), and tetramethylurea), sulfones (e.g., sulfolane), and any combination thereof in any ratio.

Examples of the organic solvent other than a haloacetic acid include preferably one or more (preferably one or two, and more preferably one) organic solvents selected from nitriles, carboxylic acids, and amides, and more preferably nitriles.

From the same viewpoints as described above, specific examples of the organic solvent other than a haloacetic acid include preferably one or more (preferably one or two, and more preferably one) organic solvents selected from acetonitrile, propionitrile, acetic acid, propionic acid, N,N-dimethylformamide (DMF), and N,N-dimethylacetamide (DMAC), and more preferably acetonitrile.

The amount of the organic solvent other than a haloacetic acid used in the reaction is not particularly limited as long as the reaction system can be sufficiently stirred. From the viewpoints of yield, suppression of by-products, economic efficiency, etc., however, when the organic solvent other than a haloacetic acid is used, the lower limit of the amount of the organic solvent other than a haloacetic acid used is, for example, over 0 (zero) liter, preferably 0.3 liters or more, or 0.4 liters or more, more preferably 0.5 liters or more, and further preferably 0.8 liters or more, or 1.0 liters or more based on 1 mol of the compound of the formula (1) (raw material).

From the same viewpoints as described above, when the organic solvent other than a haloacetic acid is used, the upper limit of the amount of the organic solvent other than a haloacetic acid is, for example, based on 1 mol of the compound of the formula (1) (raw material),
from the same viewpoints as described above, in another embodiment, the upper limit of the amount of the haloacetic acid used is, for example, 10.0 liters or less, preferably 5.0 liters or less, more preferably 3.0 liters or less, and further preferably 2.0 liters or less based on 1 mol of the compound of the formula (1) (raw material).

From the same viewpoints as described above, when the organic solvent other than a haloacetic acid is used, the range of the amount of the haloacetic acid used is an appropriate and optional combination of any of the lower limits and the upper limits described above. Examples of the combination of the upper limit and the lower limit include, but are not limited to, the following: The amount of the haloacetic acid used is, for example, 0.3 liters or more and 5.0 liters or less, and preferably 0.5 liters or more and 3.0 liters or less, or 0.8 liters or more and 2.0 liters or less based on 1 mol of the compound of the formula (1) (raw material).

When a combination of two or more reaction solvents is used, the ratio of the two or more reaction solvents may be any ratio as long as the reaction proceeds.

### (Reaction Temperature)

The reaction temperature is not particularly limited. From the viewpoints of yield, suppression of by-products, economic efficiency, etc., however, the reaction temperature is in a range of any combination of the following lower limits and upper limits: In one embodiment, the reaction temperature is, for example, 0 (zero) ° C. to 100° C., or 10° C. to 100° C., preferably 30° C. to 100° C., more preferably 30° C. to 80° C., further preferably 40° C. to 80° C., further preferably 40 to 70° C., further preferably 40° C. to 65° C., and further preferably 40° C. to 60° C. In another embodiment, the reaction temperature is, for example, 40° C. to 100° C., preferably 45° C. to 100° C., more preferably 45° C. to 80° C., further preferably 45° C. to 70° C., and further preferably 45° C. to 60° C.

### (Reaction Time)

The reaction time is not particularly limited. The reaction time can be suitably adjusted by a person skilled in the art. From the viewpoints of yield, suppression of by-products, economic efficiency, etc., however, in one embodiment, the reaction time is, for example, 5 minutes to 48 hours, preferably 10 minutes to 24 hours, more preferably 30 minutes to 24 hours, further preferably 30 minutes to 12 hours, further preferably 30 minutes to 6 hours, further preferably 30 minutes to 4 hours, and further preferably 30 minutes to 2 hours. In another embodiment, the reaction time is, for example, 30 minutes to 48 hours, preferably 1 hour to 48 hours, more preferably 1 hour to 24 hours, further preferably 1 hour to 12 hours, further preferably 1 hour to 6 hours, further preferably 1 hour to 4 hours, and further preferably 1 hour to 2 hours. As the range of the reaction time, any of the lower limits and the upper limits of the above-described ranges may be combined, and any combination of the lower limits and the upper limits of the above-described ranges is within the scope of the present invention.

### (Addition Time, Aging Time, and Reaction Time)

Herein, “aging time” refers to stirring time after completing the addition of the raw material and/or the reactant (e.g., hydrogen peroxide, and the acidic compound). When the “batch addition” is employed as the method for adding the raw material, the reactants and the like, the “reaction time” corresponds to the “aging time”.

When the raw material and/or the reactants and the like are added over a prescribed period of time, the “addition time” refers to time from the start of the addition of the raw material and/or the reactants such as hydrogen peroxide to the completion of the addition of the whole amounts thereof. Also in this case, the “aging time” corresponds to stirring time after completing the addition of the raw material and/or the reactants. In this case, it is estimated that the reaction starts after starting the addition, and the “reaction time” is a sum total of the “addition time” and the “aging time”.

### (Embodiments of Reaction)

The present reaction can be performed by a batch method using a reaction kettle, or alternatively, can be performed through a flow reaction using a continuous reactor. The continuous reactor refers to a reactor used for causing raw material supply and the reaction to continuously and simultaneously proceed. An example of the continuous reactor includes a flow reactor. A flow reactor is a reactor capable of performing reaction continuously with a raw material continuously supplied thereto. A flow reactor is roughly divided into a tubular flow reactor (including a tube flow reactor), and a tank flow reactor, both of which can perform a reaction by a continuous method. The flow reactor of the present invention may be provided with temperature control means for controlling the temperature of the flow reactor, and may be provided with, for example, a temperature control unit for heating and cooling. The temperature control unit may be any suitable unit, and examples of the temperature control unit include a bath and a jacket. The bath and the jacket may be in any suitable form. Besides, the material of the flow reactor is not particularly limited as long as it is unaltered by a raw material and a solvent, and examples include metals (e.g., titanium, nickel, stainless steel, and Hastelloy C), resins (e.g., fluororesin), glass, and porcelain (e.g., ceramics).

It is not excluded that the continuous reaction of the present invention is performed with a tank flow reactor. A preferred example of the flow reactor includes, however, a tubular flow reactor. The tubular flow reactor of the present invention may be any reactor capable of causing a liquid or a vapor-liquid mixture to continuously flow therethrough, and the cross-sectional shape of the tube may be any one of circular, rectangular, polygonal, and elliptical tubular shapes, or a shape of a combination of these shapes. Besides, the material of the tube is not particularly limited as long as it is unaltered by a raw material and a solvent, and examples include metals (e.g., titanium, nickel, stainless steel, and Hastelloy C), resins (e.g., fluororesin), glass, and porcelain (e.g., ceramics), and preferably, fluororesin (e.g., Teflon (registered trademark)) is preferred. Also the tubular flow reactor of the present invention may be provided with temperature control means for controlling the temperature, and may be provided with, for example, a temperature control unit for heating and cooling. The temperature control unit may be any suitable unit, and examples of the temperature control unit include a bath and a jacket. The bath and the jacket may be in any suitable form. As such a flow reactor, for example, spiral, shell-and-tube, and plate heat exchanger reactors can be used.

A layout method for the tube in the tubular flow reactor of the present invention is not particularly limited, and for example, may be linear layout, curved layout, or coil layout. A preferred example of the layout method includes a tubular reactor having a tube in a coil layout. Besides, the number of tube may be one, or a plurality of two or more tubes may be regularly or irregularly bundled at appropriate intervals. Herein, a tubular flow reactor having one tube is used in the description for convenience, and if production efficiency is desired to be increased, a tubular flow reactor in which a plurality of two or more tubes are regularly or irregularly bundled at appropriate intervals may be used in accordance with the description provided herein.

Besides, the tubular flow reactor of the present invention may include a mixer as desired. The mixer is not particularly limited as long as it has a function capable of continuously mixing two or more fluids, such as a gas and a liquid, or a liquid and a liquid, and examples include a Y-shaped mixer, a T-shaped mixer, and a pipeline mixer (line mixer including a static mixer). A line mixer including a static mixer or the like may be a tubular flow reactor.

### (Reaction by Flow Method)

When the flow method is employed, a mixture of prescribed amounts of the compound (1), an acidic compound, hydrogen peroxide and a solvent (with another component added if necessary) is caused to flow through a tubular reactor for causing a reaction. In this case, it is preferable that the tubular reactor to be used has a heater, and that the mixture is caused to flow through the reaction tube heated to a prescribed temperature. The reaction temperature is not particularly limited. From the viewpoint of yield, suppression of by-products, economic efficiency, etc., the reaction temperature is in the range of, for example, 0° C. (zero) to 120° C., and preferably 30° C. to 100° C.

The equivalent diameter of the tube in the tubular reactor of the present invention is not particularly limited as long as a liquid or vapor-liquid mixture can continuously flow therethrough, and also from the viewpoint of production efficiency, it is preferably 0.5 mm or more. A preferred example of the equivalent diameter includes 0.5 mm to 50 mm, and preferably about 0.5 mm to 30 mm.

The “equivalent diameter (De)” of the present invention is a value defined in accordance with the following equation:

$$De = 4 \cdot Af / Wp$$

wherein Af indicates a tube cross-sectional area, and Wp indicates a wetted perimeter.

For example, the equivalent diameter of a circular tube having a radius r is:

$$\begin{matrix}De = 4 \cdot {πr}^{2} / 2 πr \\ = 2 r\end{matrix}$$

The length of the tube of the tubular flow reactor of the present invention is not particularly limited as long as a raw material compound can be heated and sufficiently reacted therein. The length is, for example, 1 m or more, and preferably in the range of 5 m to 80 m. In order to efficiently perform the process of the present invention, since it is necessary to cause a reaction at a prescribed temperature, and/or for ensuring a sufficient reaction time, the length is, but is not limited to, preferably 5 m or more in general.

The flow rate in the flow reactor, preferably in the tubular flow reactor of the present invention depends on the equivalent diameter of the tube, and is usually 0.01 mL/min or more, and preferably 0.05 mL/min or more.

The pressure within the tubular flow reactor is, but is not limited to, for example, 0.1 MPa to 10 MPa, and preferably 0.3 MPa to 5 MPa.

### (Working-Up; Isolation and Purification)

The compounds of the formula (2), especially pyroxasulfone (2-a), which is the target product, can be isolated and purified from the reaction mixture by methods known to a person skilled in the art (e.g., extraction, washing, crystallization including recrystallization, crystal washing and/or other procedures) and improved methods thereof, and any combination thereof.

Hereinafter, the present invention will be described in more detail by Examples, but the present invention is not limited in any way by these Examples.

In the present description, the following instruments and conditions were used for the determination of physical properties and yields in Examples, Comparative Examples and Reference Examples. In addition, the products obtained in the present invention are known compounds, and were identified in the usual manner known to a person skilled in the art.

### (Measurement of pH)

Instrument: as a glass electrode type hydrogen ion concentration meter, HM-20P manufactured by DKK-TOA CORPORATION or any equivalent thereto

### (HPLC Analysis: High Performance Liquid Chromatography Analysis)

### (HPLC Analysis Conditions)

Instrument: LC 2010 Series manufactured by Shimadzu Corporation or any equivalent thereto
Column: YMC-Pack, ODS-A, A-312 (150 mm×6.0 mm ID, S-5 μm, 120A)

### Eluent:

TABLE 1

| Time | Acetonitrile | 0.1% Aqueous phosphoric |
|---|---|---|
| (min) | (%) | acid solution (%) |
| 0 | 45 | 55 |
| 10 | 45 | 55 |
| 15 | 80 | 20 |
| 20 | 80 | 20 |

Flow rate: 1.0 ml/min
Detection: UV 230 nm
Column temperature: 40° C.
Injection volume: 5 μL

The following documents can be referred to for the HPLC analysis method, as desired.
Literature (a): “Shin Jikkenkagaku Koza 9 (A New Course in Experimental Chemistry Course 9) Bunsekikagaku II (Analytical Chemistry II)”, pages 86 to 112 (1977), edited by the Chemical Society of Japan, published by Shingo Iizumi, Maruzen Co., Ltd.
Literature (b): “Jikkenkagaku Koza 20-1 (A Course in Experimental Chemistry 20-1), Bunseki Kagaku (Analytical Chemistry)”, 5th edition, pages 130 to 151 (2007), edited by the Chemical Society of Japan, published by Seishiro Murata, Maruzen Co., Ltd.

### (Yield and Purity)

Unless otherwise specified, the yield in the present invention can be calculated from the number of moles of the obtained target compound with respect to the number of moles of the raw material compound (starting compound).

That is, the term “yield” means “molar yield”.

Thus, the yield is represented by the following equation:

Yield (%)=(the number of moles of the target compound obtained)/(the number of moles of the starting compound)×100.

However, for example, in the evaluation of the reaction yield of the target product, the yield of impurities, the purity of the product, etc., HPLC area percentage analysis or GC area percentage analysis may be employed.

Herein, room temperature and ordinary temperature are from 10° C. to 30° C. Herein, “RT”, “rt”, “r.t” and “r.t.” means room temperature.

Herein, the term “overnight” means from 8 hours to 16 hours.

Herein, the procedure of “age/aged/aging” includes stirring a mixture by the usual manner known to a person skilled in the art.

### Example 1

### Production of 3-[(5-difluoromethoxy-1-methyl-3-trifluoromethylpyrazol-4-yl)methylsulfonyl]-4,5-dihydro-5,5-dimethylisoxazole

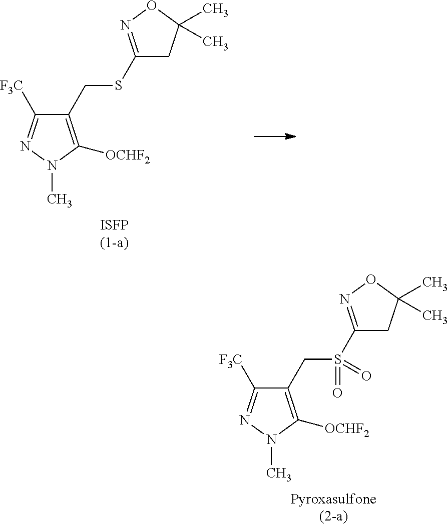 [SMILES: Cn1nc(C(F)(F)F)c(CSC2=NOC(C)(C)C2)c1OC(F)F ; Cn1nc(C(F)(F)F)c(CS(=O)(=O)C2=NOC(C)(C)C2)c1OC(F)F]

Under a nitrogen stream, the compound (1-a) (0.90 g, purity: 100%, 2.5 mmol, 100 mol %), dichloroacetic acid (5.85 g, 45.4 mmol, 1815 mol %, 1.5 L/mol), and a 35% aqueous hydrogen peroxide solution (0.69 g, 7.13 mmol, 285 mol %, containing 0.45 g (0.18 L/mol) of water) were added to a reaction flask, followed by stirring at an internal temperature of 50° C. to 55° C. for aging for 1 hour. The resultant mixture was homogeneous from the start to the end of the reaction.

At this point of time, 3-[(5-difluoromethoxy-1-methyl-3-trifluoromethylpyrazol-4-yl)methylsulfinyl]-4,5-dihydro-5,5-dimethylisoxazole (Compound 3-a; SO derivative), which is a reaction intermediate, was 0% (HPLC area percentage; 230 nm).

Acetonitrile was added to the reaction mixture. As a result of analysis by the HPLC external standard method, the target product (2-a) was obtained with a yield of 88%.

### Example 2

### Production of 3-[(5-difluoromethoxy-1-methyl-3-trifluoromethylpyrazol-4-yl)methylsulfonyl]-4,5-dihydro-5,5-dimethylisoxazole

The reaction formulas are the same as those of Example 1.

Under a nitrogen stream, the compound (1-a) (0.90 g, purity: 100%, 2.5 mmol, 100 mol %), dichloroacetic acid (5.85 g, 45.4 mmol, 1815 mol %, 1.5 L/mol), 98% sulfuric acid (0.025 g, 0.25 mmol, 10 mol %), and a 35% aqueous hydrogen peroxide solution (0.69 g, 7.13 mmol, 285 mol %, containing 0.45 g (0.18 L/mol) of water) were added to a reaction flask, followed by stirring at an internal temperature of 50° C. to 55° C. for aging for 1 hour. The resultant mixture was homogeneous from the start to the end of the reaction.

At this point of time, 3-[(5-difluoromethoxy-1-methyl-3-trifluoromethylpyrazol-4-yl)methylsulfinyl]-4,5-dihydro-5,5-dimethylisoxazole (Compound 3-a; SO derivative), which is a reaction intermediate, was 0% (HPLC area percentage; 230 nm).

Acetonitrile was added to the reaction mixture. As a result of analysis by the HPLC external standard method, the target product (2-a) was obtained with a yield of 87%.

### Example 3

### Production of 3-[(5-difluoromethoxy-1-methyl-3-trifluoromethylpyrazol-4-yl)methylsulfonyl]-4,5-dihydro-5,5-dimethylisoxazole

The reaction formulas are the same as those of Example 1.

Under a nitrogen stream, the compound (1-a) (0.90 g, purity: 100%, 2.5 mmol, 100 mol %), dichloroacetic acid (1.95 g, 15.1 mmol, 605 mol %, 0.5 L/mol), acetonitrile (1.97 g, 1.0 L/mol), and a 35% aqueous hydrogen peroxide solution (0.69 g, 7.13 mmol, 285 mol %, containing 0.45 g (0.18 L/mol) of water) were added to a reaction flask, followed by stirring at an internal temperature of 50° C. to 55° C. for aging for 12 hours. The resultant mixture was homogeneous from the start to the end of the reaction.

At this point of time, 3-[(5-difluoromethoxy-1-methyl-3-trifluoromethylpyrazol-4-yl)methylsulfinyl]-4,5-dihydro-5,5-dimethylisoxazole (Compound 3-a; SO derivative), which is a reaction intermediate, was 0% (HPLC area percentage; 230 nm).

Acetonitrile was added to the reaction mixture. As a result of analysis by the HPLC external standard method, the target product (2-a) was obtained with a yield of 89%.

### Example 4

### Production of 3-[(5-difluoromethoxy-1-methyl-3-trifluoromethylpyrazol-4-yl)methylsulfonyl]-4,5-dihydro-5,5-dimethylisoxazole

The reaction formulas are the same as those of Example 1.

Under a nitrogen stream, the compound (1-a) (0.90 g, purity: 100%, 2.5 mmol, 100 mol %), dichloroacetic acid (1.95 g, 15.1 mmol, 605 mol %, 0.5 L/mol), trichloroacetic acid (3.79 g, 23.2 mmol, 929 mol %, 0.93 L/mol), and a 35% aqueous hydrogen peroxide solution (0.69 g, 7.13 mmol, 285 mol %, containing 0.45 g (0.18 L/mol) of water) were added to a reaction flask, followed by stirring at an internal temperature of 50° C. to 55° C. for aging for 1 hour. The resultant mixture was homogeneous from the start to the end of the reaction.

At this point of time, 3-[(5-difluoromethoxy-1-methyl-3-trifluoromethylpyrazol-4-yl)methylsulfinyl]-4,5-dihydro-5,5-dimethylisoxazole (Compound 3-a; SO derivative), which is a reaction intermediate, was 0% (HPLC area percentage; 230 nm).

Acetonitrile was added to the reaction mixture. As a result of analysis by the HPLC external standard method, the target product (2-a) was obtained with a yield of 88%.

### Example 5

### Production of 3-[(5-difluoromethoxy-1-methyl-3-trifluoromethylpyrazol-4-yl)methylsulfonyl]-4,5-dihydro-5,5-dimethylisoxazole

The reaction formulas are the same as those of Example 1.

Under a nitrogen stream, the compound (1-a) (0.90 g, purity: 100%, 2.5 mmol, 100 mol %), trichloroacetic acid (3.79 g, 23.2 mmol, 929 mol %, 0.93 L/mol), and a 35% aqueous hydrogen peroxide solution (0.69 g, 7.13 mmol, 285 mol %, containing 0.45 g (0.18 L/mol) of water) were added to a reaction flask, followed by stirring at an internal temperature of 50° C. to 55° C. for aging for 1 hour. The resultant mixture was homogeneous from the start to the end of the reaction.

At this point of time, 3-[(5-difluoromethoxy-1-methyl-3-trifluoromethylpyrazol-4-yl)methylsulfinyl]-4,5-dihydro-5,5-dimethylisoxazole (Compound 3-a; SO derivative), which is a reaction intermediate, was 0% (HPLC area percentage; 230 nm).

Acetonitrile was added to the reaction mixture. As a result of analysis by the HPLC external standard method, the target product (2-a) was obtained with a yield of 91%.

### Example 6

### Production of 3-[(5-difluoromethoxy-1-methyl-3-trifluoromethylpyrazol-4-yl)methylsulfonyl]-4,5-dihydro-5,5-dimethylisoxazole

The reaction formulas are the same as those of Example 1.

Under a nitrogen stream, the compound (1-a) (0.90 g, purity: 100%, 2.5 mmol, 100 mol %), chloroacetic acid (3.34 g, 35.3 mmol, 1412 mol %, 0.84 L/mol), and a 35% aqueous hydrogen peroxide solution (0.69 g, 7.13 mmol, 285 mol %, containing 0.45 g (0.18 L/mol) of water) were added to a reaction flask, followed by stirring at an internal temperature of 50° C. to 55° C. for aging for 2.5 hours. At this point, crystals were precipitated, and the mixture was changed to a suspension. The resultant was further aged for 0.5 hours at the same temperature.

At this point of time, 3-[(5-difluoromethoxy-1-methyl-3-trifluoromethylpyrazol-4-yl)methylsulfinyl]-4,5-dihydro-5,5-dimethylisoxazole (Compound 3-a; SO derivative), which is a reaction intermediate, was 0% (HPLC area percentage; 230 nm).

Acetonitrile was added to the reaction mixture to dissolve the reaction mixture in a homogeneous solution. As a result of analysis by the HPLC external standard method, the target product (2-a) was obtained with a yield of 88%.

### Reference Example 1

### Reproduction Experiment of Example 4 of CN 111574511 A (Patent Literature 8)

### Production of 3-[(5-difluoromethoxy-1-methyl-3-trifluoromethylpyrazol-4-yl)methylsulfonyl]-4,5-dihydro-5,5-dimethylisoxazole (Compound 2-a)

The reaction formulas are the same as those of Example 1.

Under a nitrogen stream, the compound (1-a) (0.90 g, purity: 100%, 2.5 mmol, 100 mol %), methanol (2.97 g, 1.5 L/mol), 98% sulfuric acid (0.023 g, 0.225 mmol, 9 mol %), and a 30% aqueous hydrogen peroxide solution (0.81 g, 7.12 mmol, 285 mol %, containing 0.57 g (0.2 L/mol) of water) were added to a reaction flask, followed by stirring at room temperature for aging for 6 hours.

At this point of time, 3-[(5-difluoromethoxy-1-methyl-3-trifluoromethylpyrazol-4-yl)methylsulfinyl]-4,5-dihydro-5,5-dimethylisoxazole (Compound 3-a; SO derivative), which is a reaction intermediate, was 13.97% (HPLC area percentage; 230 nm).

Acetonitrile was added to the reaction mixture to dissolve the reaction mixture in a homogeneous solution. As a result of analysis by the HPLC external standard method, the yield was 0%, and the target product (2-a) was not obtained. This process is not reproducible.

All publications, patents, and patent applications described herein are hereby fully incorporated by reference in their entirety for the purpose of describing and disclosing the methods described in those publications, patents, and patent applications that may be used in connection with the description herein. To the extent necessary to understand or complete the disclosure of the present invention, all publications, patents, and patent applications described herein are expressly incorporated herein by reference to the same extent as if each were individually incorporated. All publications, patents, and patent applications discussed above and throughout this specification are provided solely for disclosure prior to the filing date of this application.

Any processes and reagents similar or equivalent to those described herein can be employed in the practice of the present invention. Accordingly, the present invention is not to be limited by the foregoing description, but is intended to be defined by the claims and their equivalents. Those equivalents fall within the scope of the present invention as defined by the appended claims.

### INDUSTRIAL APPLICABILITY

As disclosed in Patent Document 1, a compound of the general formula (2) (sulfone derivative: SO₂ derivative) has excellent herbicidal activity. According to the present invention, an industrially favorable novel production process for the compound of the general formula (2) useful as a herbicide is provided.

As described above herein, the process of the present invention is economical, is environmentally friendly, and is highly industrially variable. In particular, in the process of the present invention, the ratio of a compound of the formula (3) (sulfoxide derivative: SO derivative) in a product is sufficiently low. Here, the compound of the formula (3) (sulfoxide derivative: SO derivative) is an intermediate of an oxidation reaction, and can be a cause of reduced quality as a herbicide and crop injury. In addition, a reproducible and practicable process has been provided by the present invention. Accordingly, the present invention is highly industrially applicable.

1. A process for producing a compound of formula (2), comprising reacting a compound of formula (1) with an oxidizing agent in the presence of a haloacetic acid and in the absence of a transition metal: 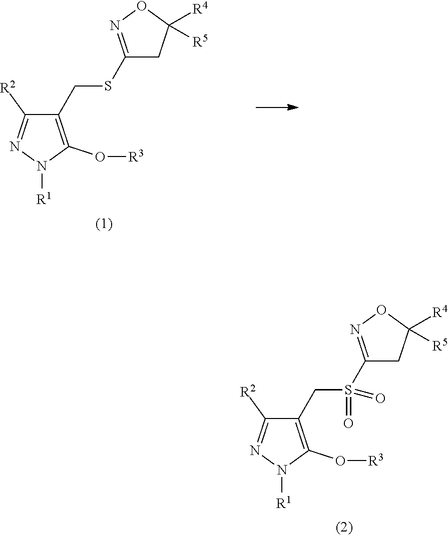
wherein in the formula (1) and the formula (2),
R¹, R², and R³ are each independently a (C1-C6)alkyl optionally substituted with one or more substituents; a (C3-C6)cycloalkyl optionally substituted with one or more substituents; a (C2-C6)alkenyl optionally substituted with one or more substituents; a (C2-C6)alkynyl optionally substituted with one or more substituents; or a (C6-C10)aryl optionally substituted with one or more substituents, and
R⁴ and R⁵ are each independently a (C1-C6)alkyl optionally substituted with one or more substituents; a (C3-C6)cycloalkyl optionally substituted with one or more substituents; a (C2-C6)alkenyl optionally substituted with one or more substituents; a (C2-C6)alkynyl optionally substituted with one or more substituents; a (C1-C6)alkoxy optionally substituted with one or more substituents; or a (C6-C10)aryl optionally substituted with one or more substituents, or
R⁴ and R⁵, together with a carbon atom to which they are attached, form a 4- to 12-membered carbocyclic ring, wherein the carbocyclic ring is optionally substituted with one or more substituents.

2. The process according to claim 1, wherein the haloacetic acid is selected from monochloroacetic acid, dichloroacetic acid, and trichloroacetic acid.

3. The process according to claim 1, wherein the haloacetic acid is dichloroacetic acid.

4. The process according to claim 1, wherein the haloacetic acid is trichloroacetic acid.

5. The process according to any one of claims 1 to 4, wherein the oxidizing agent is hydrogen peroxide.

6. The process according to any one of claims 1 to 5,
wherein in the formula (1) and the formula (2),
R¹ is a (C1-C4)alkyl,
R² is a (C1-C4)perfluoroalkyl,
R³ is a (C1-C4)alkyl optionally substituted with 1 to 9 fluorine atoms, and
R⁴ and R⁵ are each independently a (C1-C4)alkyl.

7. The process according to any one of claims 1 to 5,
wherein in the formula (1) and the formula (2),
R¹ is methyl,
R² is trifluoromethyl,
R³ is difluoromethyl, and
R⁴ and R⁵ are methyl.
